(19) United States
(12) Patent Application Publication
(10) Pub. No.: US 2019/0116725 A1
(43) Pub. Date: Apr. 25, 2019

(54) Fertilization Map Generation Method, Fertilization Map Generation System, Fertilization Map Generation Device, and Fertilization Map Generation Program

(71) Applicant: Topcon Corporation, Tokyo (JP)

(72) Inventors: Issei HANYA, Itabashi-ku, Tokyo (JP); Shinkai SHU, Itabashi-ku, Tokyo (JP); Peng ZHAO, Itabashi-ku, Tokyo (JP); Shugo AKIYAMA, Itabashi-ku, Tokyo (JP); Hiroshi OKAMOTO, Sapporo-shi, Hokkaido (JP); Keisuke HARA, Sapporo-shi, Hokkaido (JP)

(73) Assignee: Topcon Corporation, Tokyo (JP)

(21) Appl. No.: 16/090,223

(22) PCT Filed: Mar. 31, 2017
(86) PCT No.: PCT/JP2017/013539

§ 371 Date: Sep. 29, 2018

(30) Foreign Application Priority Data
Apr. 1, 2016 (JP) 2016-074654

(51) Int. Cl. A01C 21/00; G01N 33/02; G01S 19/13; G06Q 50/02; G06Q 10/06

(52) U.S. Cl. CPC A01C 21/007; A01C 21/005; G06Q 10/06315; G01S 19/13; G06Q 50/02; G01N 33/025

(57) ABSTRACT

A fertilization map creating system includes a growth sensor, a GPS device, and a growth information accumulating part that stores data, a fertilization map being created based on growth data and position data stored in the growth information accumulating part, and the growth information accumulating part storing the position data and the growth data while a tractor drives in a field. The system further includes a growth condition calculation part that obtains a growth condition of previously set each area in the field based on the data stored in the memory; a fertilization amount calculation part that obtains an amount of fertilization of each area based on the growth condition of each area; and a map creating part that creates the fertilization map showing the amount of fertilization of each area obtained by the fertilization calculation part.

### CROSS-REFERENCE TO RELATED APPLICATION

This application is based upon and claims the benefit of priority from Japanese Patent Application No. 2016-074654 filed on Apr. 1, 2016, the entire disclosure of which is incorporated herein by reference.

### TECHNICAL FIELD

The present disclosure relates to a fertilization map creating method, a fertilization map creating system, a fertilization map creating apparatus, and a fertilization map creating program.

### BACKGROUND ART

A field is usually fertilized before being seeded to improve soil suitable for crops.

The field is fertilized with a fertilizer spreading device equipped in a tractor, for example (see JP2012-247235A).

### SUMMARY

### Technical Problem

A field has both a good growth area and a poor growth area. The good growth area requires the reduced amount of fertilizer and the poor growth area requires the increased amount of fertilizer. The good growth area and the poor growth area as well as the amount of fertilizer are determined based on past experiences. No sprout on a field before being seeded makes it difficult to accurately determine the good growth area and the poor growth area. Accordingly, such a field is excessively fertilized over a wide area.

It is, therefore, an object of this disclosure to provide a fertilization map creating method, a fertilization map creating system, a fertilization map creating apparatus, and a fertilization map creating program that create a fertilization map accurately showing a good growth area and a poor growth area as well as the appropriate amount of fertilizer according to that area.

### Solution to Problem

A fertilization map creating method of this disclosure includes: obtaining a growth condition of previously set each area in a field for each measurement time based on past growth data in each position in the field; calculating an amount of fertilization of the each area to be fertilized before being seeded according to the growth condition of the each area for the each measurement time; and creating a fertilization map showing the amount of fertilization of the each area in the field based on the amount of fertilization.

A fertilization map creating system of this disclosure includes: a growth sensor and a GPS device equipped in a tractor; a memory that stores, as past data, growth data detected by the growth sensor and position data of the tractor detected by the GPS device, a fertilization map being created based on the growth data and the position data stored in the memory, the memory storing the positon data and the growth data of each position in a field while the tractor drives in the field; a growth condition calculation part that obtains a growth condition of previously set each area in the field based on the growth data of the each position stored in the memory; a fertilization amount calculation part that obtains an amount of fertilization of the each area to be fertilized before being seeded based on the growth condition of the each area obtained by the growth condition calculation part; and a map creating part that creates the fertilization map showing the amount of fertilization of the each area obtained by the fertilization amount calculation part.

A fertilization map creating apparatus of this disclosure includes: a memory that stores past growth data of each position in a field, a fertilization map being created based on the growth data of the each position stored in the memory; a growth condition calculation part that obtains a growth condition of previously set each area in the field based on the growth data of the each position stored in the memory; a fertilization amount calculation part that calculates an amount of fertilization of the each area to be fertilized before being seeded based on the growth condition of the each area obtained by the growth condition calculation part; and a map creating part that creates the fertilization map showing the amount of fertilization of the each area obtained by the fertilization amount calculation part.

A fertilization map creating program of this disclosure operates a computer creating a fertilization map showing an amount of fertilization of each area according to a growth condition of the each area in a field as: a growth condition calculator that obtains the growth condition of previously set each area in the field based on growth data of each position in the field, the growth data being stored in a memory; a fertilization amount calculator that calculates an amount of fertilization of the each area to be fertilized before being seeded based on the growth condition of the each area obtained by the growth condition calculator; and a map creator that creates the fertilization map showing the amount of fertilization of the each area obtained by the fertilization amount calculator.

### BRIEF DESCRIPTION OF DRAWINGS

FIG. 1 is a block diagram illustrating a configuration of one embodiment of a fertilization map creating system according to this disclosure.

FIG. 2 is a view illustrating how to obtain a height of a plant.

FIG. 3 is a plan view illustrating a shape of a field.

FIG. 4 is a flowchart of an operation of the fertilization map creating system.

FIG. 5 is a table showing one example of growth data classified for each area in a field and for each measurement time.

FIG. 6 is a basal fertilization plan map created by the fertilization map creating system.

FIG. 7 is a table showing standardized growth data for each area and each measurement time.

### DESCRIPTION OF EMBODIMENT

Hereinafter, an embodiment of a fertilization map creating system having a fertilization map creating apparatus executing a fertilization map creating method according to this disclosure is described with reference to the drawings.

### Embodiment

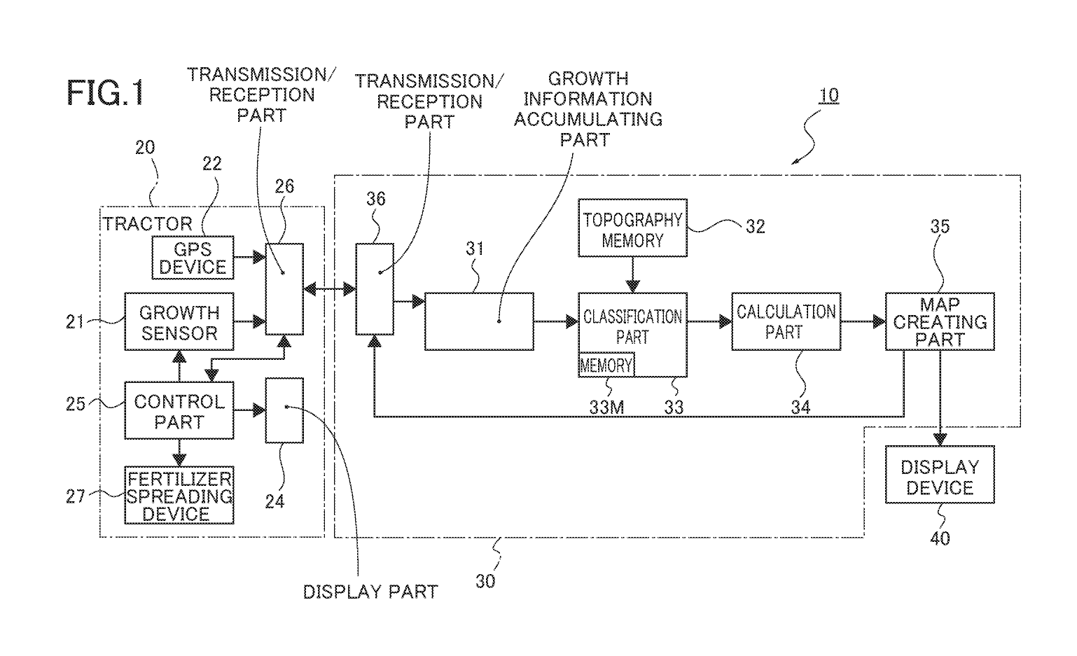

FIG. 1 illustrates a configuration of a fertilization map creating system. The fertilization map creating system 10 includes a growth sensor 21 and a GPS device 22, which are equipped in a tractor 20, a personal computer (PC) 30 as a computer, and a display device (display) 40.

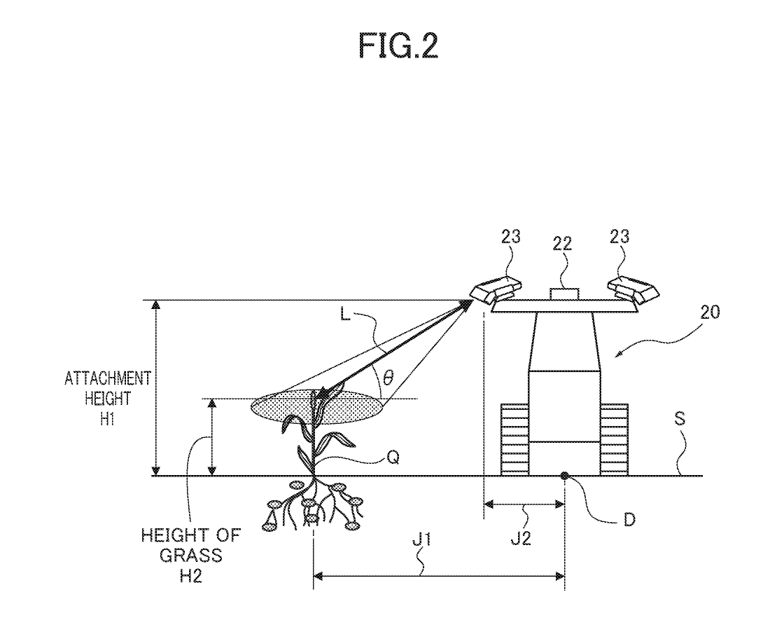

As illustrated in FIG. 2, the growth sensor 21 includes a laser ranging device 23. The growth sensor 21 measures a distance L to grass Q with the laser ranging device 23, and measures a height H2 of the grass Q based on the distance L. This height H2 is measured as a growth condition.

The height H2 is measured by the following equation.

H2=H1−L×sinθ

Where, L represents a distance to the grass Q measured by the laser ranging device 23, and H1 represents a height from a ground S to the laser ranging device 23.

In this embodiment, the laser ranging device 23 is used for the growth sensor 21. However, it is not limited thereto. For example, the plant sensor described in JP2012-247235A may be used for the growth sensor 21.

The tractor 20 is equipped with a fertilizer spreading device 27 capable of adjusting a spreading amount and a display part 24 that displays the after-described fertilization map. The tractor 20 spreads fertilizer based on the fertilization map displayed on the display part 24. Reference number 25 denotes a control part that controls the fertilizer spreading device 27 and a transmission/reception part 26. Growth data as the growth condition detected by the growth sensor 21 and position data detected by the GPS device 22 are wirelessly sent to the personal computer 30 by the transmission/reception part 26.

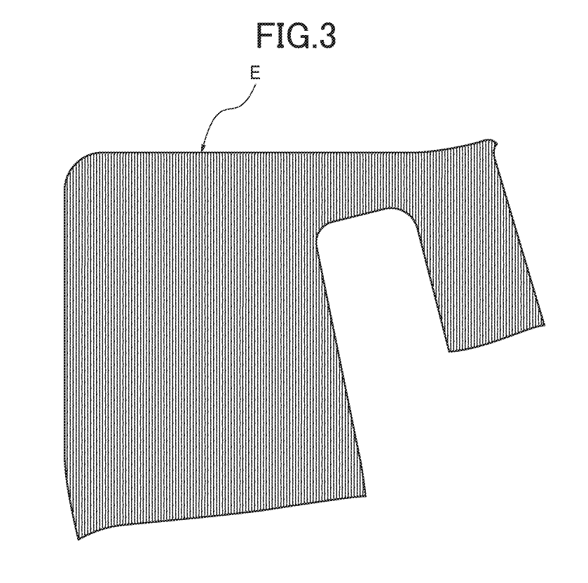

The personal computer 30 includes a transmission/reception part 36 that receives the growth data and the position data sent from the transmission/reception part 26, a growth information accumulating part (memory) 31 that stores (accumulate) the growth data and the position data received by the transmission/reception part 36, a topography memory 32 that stores a topography and a position of a field E illustrated in FIG. 3, a classification part 33, a calculation part 34, and a map creating part (map creator) 35.

Although the growth information accumulating part 31 and the topography memory 32 are provided in the personal computer 30 for simplifying the description, for example, data base on cloud is actually used through the Internet.

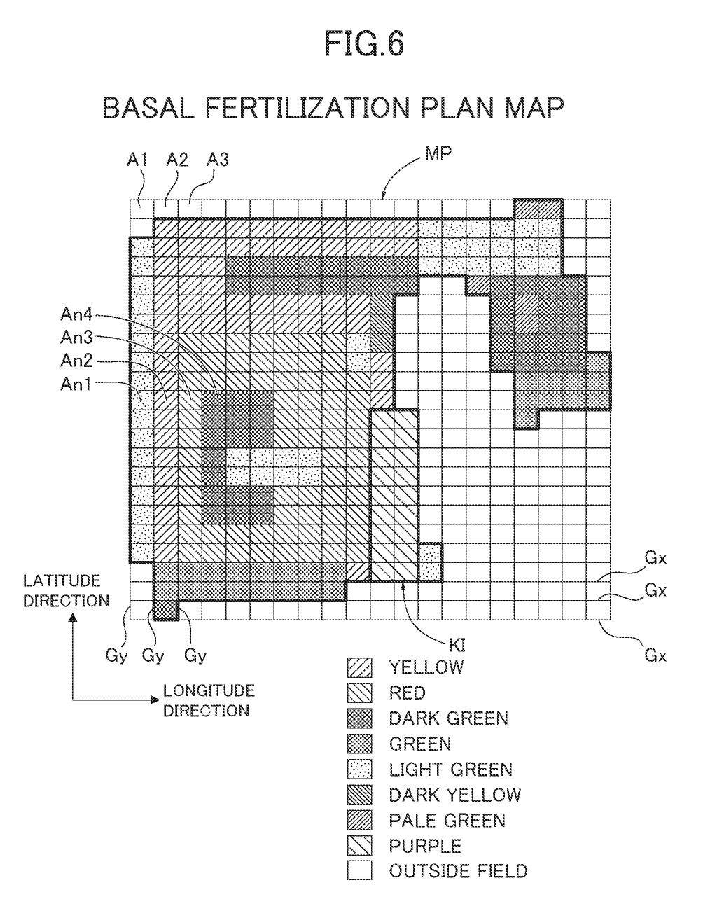

The classification part 33 classifies the topography data stored in the topography memory 32 into a plurality of areas as illustrated in FIG. 6, and classifies the position data and the growth data stored in the growth information accumulating part 31 for each area corresponding to the position data and for each measurement time (past) to store the growth data in a memory 33M for each area. Namely, the growth data is stored in the memory 33M for each area and each measurement time.

The calculation part 34 calculates an average value of the growth data in the area for each area in each measurement time, and standardizes the average value of each area for each measurement time. Namely, the growth condition of each area is obtained (measured) for each measurement time. Further, an average value (growth condition) of the values standardized for each measurement time is measured for each area, and the amount of fertilizer for basal fertilization for each area is calculated based on the growth condition which is the average value calculated for each area.

The calculation part 34 operates as a growth condition calculation part (growth condition calculator) that obtains (measures) a growth condition of each area and a fertilization amount calculation part (fertilization amount calculator) that calculates the amount of fertilization for each area based on the growth condition.

The map creating part 35 creates a fertilization map as a distribution diagram showing the amount of fertilization for each area calculated by the calculation part 34.

The created fertilization map is displayed on the display device 40, and the data of the fertilization map is sent to the tractor 20 by the transmission/reception part 36.

The growth information accumulating part 31, the classification part 33, the calculation part 34, and the map creating part 35 constitute the fertilization map creating apparatus that creates the fertilization map.

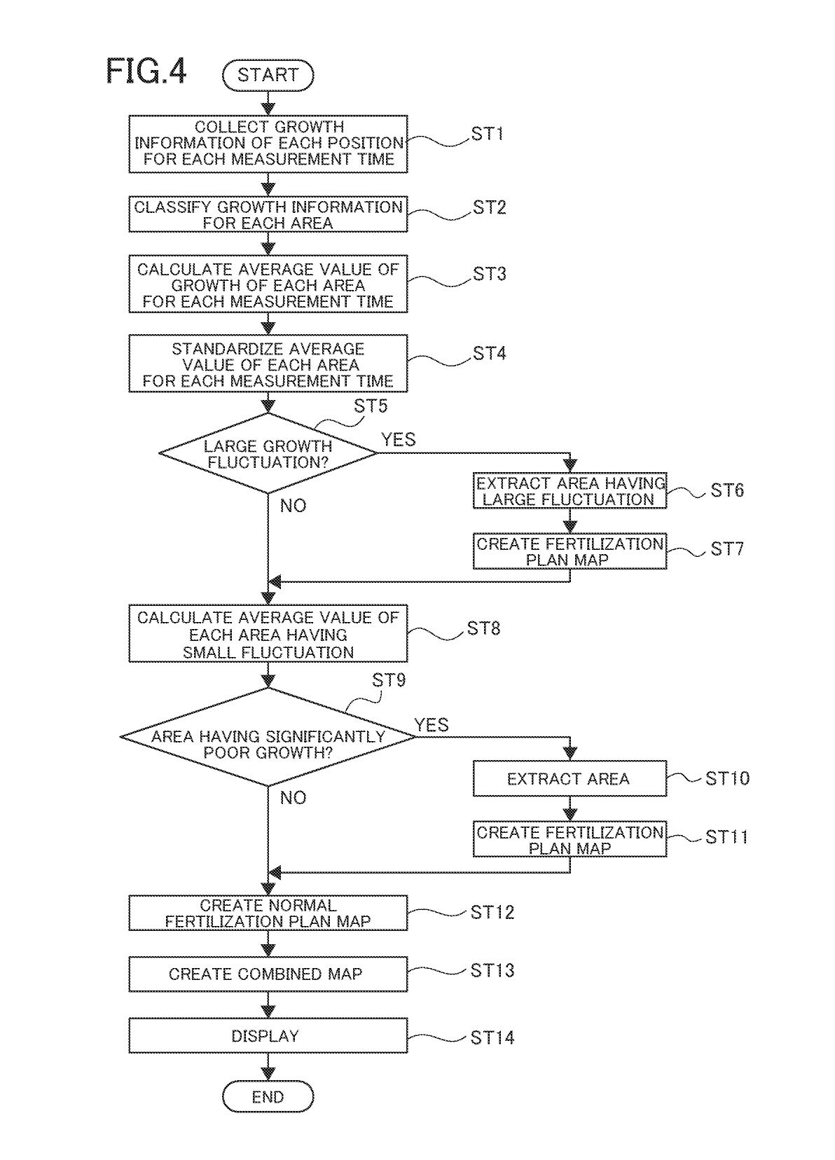

The data of the fertilization map is stored in a not-shown memory of the tractor 20, and the fertilization map is displayed on the display 24 of the tractor 20. The display 24 displays the position of the tractor 20 on the fertilization map based on the position data detected by the GPS device. Next, the operation of the fertilization map creating system 10 constructed as described above is described with reference to the flowchart of FIG. 4. In addition, the flowchart shows the process operation of the map creating program that creates the fertilization map.

In Step 1, the growth data and the position data detected by the growth sensor 21 and the GPS device 22 are collected while the tractor 20 drives in the field E. The collected data is loaded in the growth information accumulating part 31 of the personal computer 30. The growth information accumulating part 31 stores, as a pair, the growth data detected by the growth sensor 21 and the position data detected by the GPS device 22 at the detection.

As illustrated in FIG. 2, although the position of the tractor 20 is shifted from the position of the grass Q having the height H2 detected by the growth sensor 21, a distance J1 from the position of the tractor 20 to the position of the grass Q is accurately measured because a distance J2 from an attachment position of the laser ranging device 23 to a position D (attachment position of GPS device 22 in horizontal direction) of the tractor 20 is known. The position of the grass Q is herein described as the position D of the tractor 20 for simplifying the description.

The collected data includes the growth data and the position data of a crop U1 firstly (for example, spring) cultivated in the field E (hereinafter referred to as first crop), the growth data and the position data of a crop U2 secondly (for example, fall) cultivated in the field E (hereinafter referred to as second crop), and the growth data and the position data of a crop U3 thirdly (for example, next spring) cultivated in the field E (hereinafter referred to as third crop). The crops U1 to U3 are different crops, respectively.

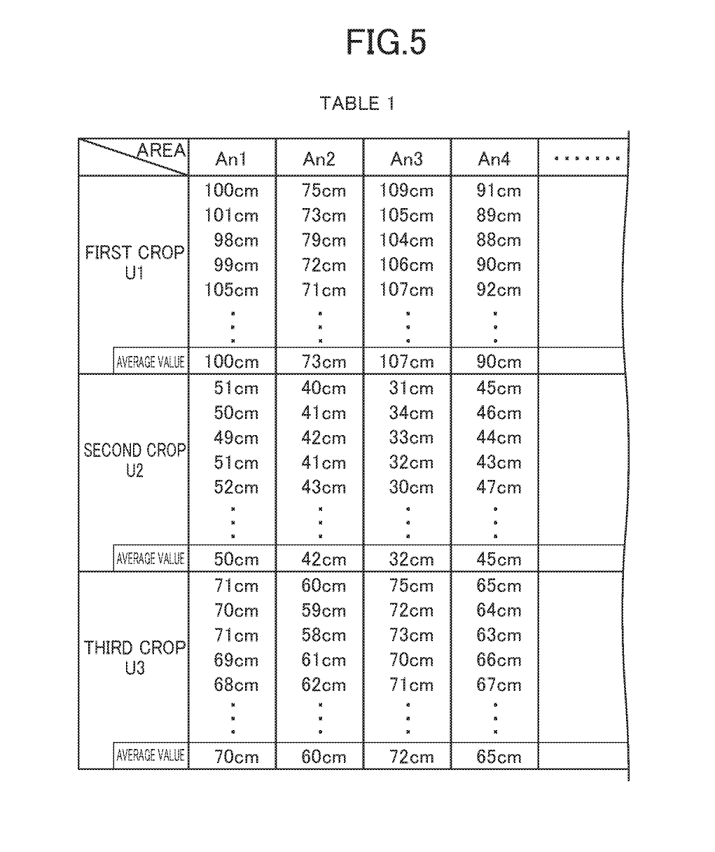

In Step 2, the growth data is classified for each area in the field E based on the position data stored in the growth information accumulating part 31. For example, as shown in Table 1 of FIG. 5, as to the first crop U1, all the growth data detected in the areas An1, An2, An3 . . . is recoded in each section of each area An1, An2, An3 . . . . Similarly, as to the second and third crops U2, U3, all the growth data detected in the areas An1, An2, An3 . . . is recorded in each section of each area An1, An2, An3 . . . . Namely, the growth data is classified for each measurement time and each area to be stored.

As illustrated in FIG. 6, for example, a plurality of areas A1, A2, A3 . . . An1, An2, An3, An4 . . . is defined by grid lines Gx, Gy which are drawn, in the field E, at equal intervals along the latitude direction and the longitude direction. The longitudinal size and the lateral size of each area A1, A2, A3 . . . An1, An2, An3, An4 . . . are set to, for example, 5 m, respectively.

In Step 3, the average value of the growth data in each area is calculated for each measurement time. Table 1 of FIG. 5 shows one example. Table 1 of FIG. 5 shows the average value of each area An1, An2, An3, An4 . . . of the first to third crops U1 to U3. The average values of the areas An1, An2, An3, An4 . . . of the first crop are 100 cm, 73 cm, 107 cm, 90 cm . . . , respectively. The average values of the areas An1, An2, An3, An4 of the second crop are 50 cm, 42 cm, 32 cm, 45 cm, respectively. The average values of the areas An1, An2, An3, An4 of the third crop are 70 cm, 60 cm, 72 cm, 65 cm, respectively.

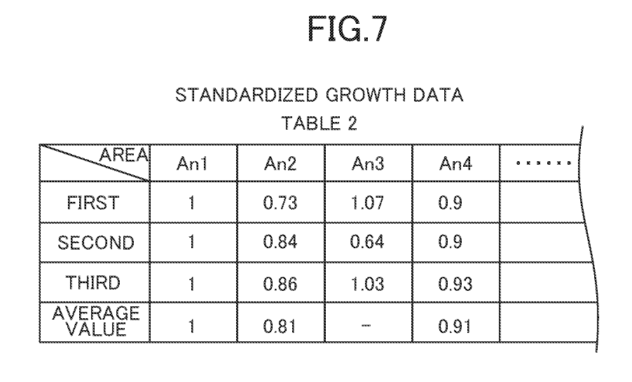

In Step 4, the average value of each area is standardized for each measurement time. For example, the average value of the entire field E is calculated based on the average value of each area for the first crop. Namely, such an average value is obtained by dividing the total of the average value of each area with the number of areas of the field E. When the average value of the entire field E for the first crop is, for example, 100 cm, the standardized value of the area An1 is 100/100=1, and the standardized values of the areas A2, A3, A4 are 73/100=0.73, 107/100=1.07, 90/100=0.9, respectively, as illustrated in Table 2 of FIG. 7.

The standardized values for the second and third crops are similarly calculated. For example, when the average value of the entire field E for the second crop is 50 cm and the average value of the entire field E for the third crop is 70 cm, the standardized values of the areas An1, An2, An3, An4 for the second crop are 1, 0.84, 0.64, 0.9, respectively, and the standardized values of the areas An1, An2, An3, An4 for the third crop are 1, 0.86, 1.03, 0.93, respectively.

Namely, in Step 4, the growth condition is obtained (measured) for each measurement time and each area.

In Step 5, it is determined whether an area has a considerably large difference in the standardized value between the first and second crops, the second and third crops, or the first and third crops. Namely, it is determined whether an area has a difference equal to or larger than a predetermined value. In this embodiment, in Table 2 of FIG. 7, the area An3 has 0.43 of the difference in the standardized value between the first and second crops. When the predetermined value is set to 0.4, for example, the process proceeds from Step 5 to Step 6.

In Step 6, the area having a difference in the standardized value equal to or larger than a predetermined value, for example, the area An3 is extracted. The area An3, which fluctuates greatly depending on season and year, is full of stones, or is a land having significantly poor drainage. The calculation part 34 extracts this area, and operates as a first extractor (first area extractor) that extracts an area having a considerably large difference in the growth condition compared to each measurement time.

In Step 7, as the area extracted at Step 6 has a significantly poor land condition, a special fertilization plan according to this condition is created. A farmer creates this fertilization plan. A fertilization plan map showing the extracted area with red, for example, is created to show the area having a significantly poor land condition.

In Step 8, the average value of three standardized values is calculated for each area having small fluctuation. In Table 2 of FIG. 7, the average value of three standardized values is calculated for each area An1, An2, An4 except for the area An3 having large fluctuation. When the process proceeds to Step 8 from Step 5, all average values for all areas are calculated.

The average value in Table 2 of FIG. 7 shows the average value of three standardized values for each area An1, An2, An4.

In Step 9, it is determined, from the average value calculated in Step 8, whether an area has significantly poor growth. Namely, it is determined whether an area has significantly poor growth for the three areas. In the case of YES, the process proceeds to Step 10. It is determined that an area having an average value of standardized values smaller than 1 by 0.15 or more, for example, has significantly poor growth. Namely, in Table 2 of FIG. 7, the area An2 is determined as an area having significantly poor growth.

In Step 10, the area having significantly poor growth is extracted. For example, this area is poorly drained and does not get much sunshine. For this reason, the growth of this area is significantly poor. The calculation part 34 extracts this area, and operates as a second extractor (second area extractor) that extracts the area having a significantly poor growth condition.

In Step 11, as the area extracted in Step 10 has a significantly poor land condition, a special fertilization plan according to this land is created. A farmer creates this fertilization plan. A fertilization map showing the extracted area with yellow, for example, is created to show an area having a significantly poor land condition.

In Step 12, a fertilization plan map according to growth of each area is created for an area where a normal growth condition is expected, namely, the areas An1, An4 in Table 2 of FIG. 7 and areas having a growth condition similar to those areas.

The fertilization plan map is created based on the comparison of the previously set parameter representing the amount of fertilization to the standardized average value of the growth data. For example, the parameter values such as 0.89 to 0.94, 0.95 to 1.05, 1.06 to 1.11 are previously set, and the amount of fertilization V1, V2, V3, V4 is set according to each of the parameter values 0.89 to 0.94, 0.95 to 1.05, 1.06 to 1.11, 1.12 to 1.17. In this case, the relationship of the amount of fertilization is V1>V2>V3>V4.

The standardized average value of the growth data of the area An1 is 1 as illustrated in Table 2 of FIG. 7, which is within the range of the parameter values 0.95 to 1.05. The amount of fertilization of the area An1 is set to V2. Similarly, the standardized average value of the growth data of the area An4 is 0.91, which is within the range of the parameter values 0.89 to 0.94. The amount of fertilization of the area An4 is set to V1.

The fertilization plan map with colors according to the amount of fertilization V1 to V4 is created. In this fertilization plan map, for example, dark green shows the amount of fertilization V1, green shows the amount of fertilization V2, light green shows the amount of fertilization V3, and pale green shows the amount of fertilization V4.

In Step 13, a basal fertilization plan map as a fertilization map is created by combining the fertilization plan maps created in Steps 7, 11, 12. For example, a basal fertilization plan map MP is created as illustrated in FIG. 6.

As described above, in the basal fertilization plan map MP, a red area shows large growth fluctuation, a yellow area shows poor growth, and a green area shows normal growth. Further, the dark green area shows slightly poor growth, which is within a normal growth range, a light green area shows good growth, and a pale green area shows better growth. The amount of fertilization increases in accordance with darkening in green. In addition, a white portion shows an area except the field E, and a purple area K1 shows, for example, a test region which is previously set.

In Step 14, the basal fertilization plan map MP shown in FIG. 6 is displayed on the display 40 and is stored in a not-shown memory.

The basal fertilization plan map MP displayed on the display device 40 shows an area having good growth and an area having poor growth in the field E, and also shows an area having considerably large growth fluctuation depending on year and season and an area having significantly poor growth. The map MP also shows the amount of fertilization for each area. Accordingly, the condition of the field E can be confirmed even before the field is seeded.

The data of the basal fertilization plan map MP stored in a memory is sent to the tractor 20 by the transmission/reception part 36. The data may be wirelessly sent or may be sent with signal lines.

When the transmission/reception part 26 of the tractor 20 receives the data of the basal fertilization plan map MP, the control part 25 stores the data of the basal fertilization plan map MP in a not-shown memory.

The basal fertilization plan map MP is displayed on the display part 24 based on the data of the basal fertilization plan map MP stored in the memory. The position of the tractor 20 is shown on the basal fertilization plan map MP of the display part 24 based on the positional information measured by the GPS device 22. An operator confirms the position of the tractor 20 on the basal fertilization plan map MP displayed on the display part 24, so as to recognize the amount of basal fertilizer for basal fertilization before seeding the area in that position. The operator thereby fertilizes that area as much as necessary.

Namely, even in the field E before being seeded, the area having good growth and the area having poor growth are clearly distinguished, and an area having considerably large growth fluctuation depending on year and season and an area having significantly poor growth are also clearly distinguished. Moreover, the appropriate amount of fertilization can be obtained in each area in the field E. The field E is thereby prevented from being excessively fertilized. As a result, the fertilizer can be saved.

The control part 25 controls the fertilizer spreading device 27 based on the basal fertilization plan map MP to automatically fertilize each area in the field E with the appropriate amount, and to dramatically improve the operation efficiency.

In the above embodiment, the growth information accumulating part 31 stores, as a pair, the growth condition detected by the growth sensor 21 and the position data detected by the GPS device 22 at the detection. In this case, the growth information accumulating part 31 may store the growth condition with respect to each area based on the position data. In this case, the classification part 33 is omitted.

In the above embodiment, the tractor 20 is equipped with the growth sensor 21 and the GPS device 22. The tractor 20 may be equipped with the fertilization map creating apparatus including the growth information accumulating part 31, the classification part 33, the calculation part 34, and the map creating part 35.

This disclosure should not be limited to the embodiment, and it should be appreciated that variations or modifications may be made in the embodiments described by persons skilled in the art without departing from the gist of this disclosure.

What is claimed is:

1. A fertilization map creating method comprising:
obtaining a growth condition of previously set each area in a field for each measurement time based on past growth data in each position in the field;
calculating an amount of fertilization of the each area to be fertilized before being seeded according to the growth condition of the each area for the each measurement time; and
creating a fertilization map showing the amount of fertilization of the each area in the field based on the amount of fertilization.

2. The fertilization map creating method according to claim 1, further comprising:
extracting an area having a considerably large difference in the growth condition compared to the each measurement time based on the growth condition of the each area for the each measurement time; and
showing the extracted area on the fertilization map.

3. The fertilization map creating method according to claim 1, further comprising:
extracting an area having significantly poor growth based on the growth condition of the each area for the each measurement time; and
showing the extracted area on the fertilization map.

4. A fertilization map creating system comprising:
a growth sensor and a GPS device equipped in a tractor;
a memory that stores, as past data, growth data detected by the growth sensor and position data of the tractor detected by the GPS device, a fertilization map being created based on the growth data and the position data stored in the memory, the memory storing the positon data and the growth data of each position in a field while the tractor drives in the field;
a growth condition calculation part that obtains a growth condition of previously set each area in the field based on the growth data of the each position stored in the memory;
a fertilization amount calculation part that obtains an amount of fertilization of the each area to be fertilized before being seeded based on the growth condition of the each area obtained by the growth condition calculation part; and
a map creating part that creates the fertilization map showing the amount of fertilization of the each area obtained by the fertilization amount calculation part.

5. The fertilization map creating system according to claim 4,
wherein the growth condition calculation part obtains the growth condition of the each area in the field for each measurement time,
the system further comprising a first area extractor that extracts an area having a considerably large difference in the growth condition compared to the each measurement time based on the obtained growth condition for the each measurement time, and
wherein the area extracted by the first area extractor is shown on the fertilization map created by the map creating part.

6. The fertilization map creating system according to claim 4, further comprising a second area extractor that extracts an area having significantly poor growth based on the growth condition of the each area obtained by the growth condition calculation part,
wherein the area extracted by the second area extractor is shown on the fertilization map.

7. The fertilization map creating system according to claim 4, further comprising a display part that displays the fertilization map created by the map creating part.

8. A fertilization map creating apparatus comprising:
a memory that stores past growth data of each position in a field, a fertilization map being created based on the growth data of the each position stored in the memory;
a growth condition calculation part that obtains a growth condition of previously set each area in the field based on the growth data of the each position stored in the memory;
a fertilization amount calculation part that calculates an amount of fertilization of the each area to be fertilized before being seeded based on the growth condition of the each area obtained by the growth condition calculation part; and
a map creating part that creates the fertilization map showing the amount of fertilization of the each area obtained by the fertilization amount calculation part.

9. The fertilization map creating apparatus according to claim 8,
wherein the growth condition calculation part obtains the growth condition of the each area in the field for each measurement time,
the apparatus further comprising a first area extractor that extracts an area having a considerably large difference in the growth condition compared to the each measurement time based on the obtained growth condition for the each measurement time, and
wherein the area extracted by the first area extractor is shown on the fertilization map created by the map creating part.

10. The fertilization map creating apparatus according to claim 8, further comprising a second area extractor that extracts an area having significantly poor growth based on the growth condition of the each area obtained by the growth condition calculation part,
wherein the area extracted by the second area extractor is shown on the fertilization map.

11. The fertilization map creating apparatus according to claim 8, further comprising a display part that displays the fertilization map created by the map creating part.

12. (canceled)
